(19) United States
(12) Patent Application Publication
(10) Pub. No.: US 2013/0034888 A1
(43) Pub. Date: Feb. 7, 2013

(54) METHODS AND COMPOSITIONS FOR DECOMPOSITION OF BIOMASS

(71) Applicants: Rajeev Aurora, Wildwood, MO (US); Shelley D. Minteer, Salt Lake City, UT (US)

(72) Inventors: Rajeev Aurora, Wildwood, MO (US); Shelley D. Minteer, Salt Lake City, UT (US)

(21) Appl. No.: 13/566,009

(22) Filed: Aug. 3, 2012

Related U.S. Application Data
(60) Provisional application No. 61/515,006, filed on Aug. 4, 2011.

(51) Int. Cl. C12P 7/16; G01N 21/64; C12P 7/40

(52) U.S. Cl. 435/136; 435/160; 436/94

(57) ABSTRACT

Disclosed are methods for detecting cellulose in cellulosic materials and producing alcohol using cellulosic materials. More particularly, disclosed are methods for producing alcohol in a cell-free system by contacting pyruvate with enzymes from a minimal enzymatic pathway. Also disclosed are methods of producing pyruvate by culturing a microorganism under hypoxic conditions. Disclosed are methods for detecting cellulose in a sample using Congo red dye.

### CROSS-REFERENCE TO RELATED APPLICATIONS

This application claims priority to provisional patent application No. 61/515,006, filed on Aug. 4, 2011, which is incorporated herein by reference in its entirety.

### BACKGROUND OF THE DISCLOSURE

The present disclosure generally relates to detecting cellulose in cellulosic materials and producing alcohol using cellulosic materials. More particularly, the present disclosure relates to methods for producing butanol from pyruvate produced from cellulosic material using a cell-free system. The present disclosure further relates to methods for producing pyruvate from cellulosic material. The present disclosure also relates to methods for detecting cellulase activity.

Extensive efforts are directed to developing renewable alternatives to fossil fuels. One alternative biofuel under consideration is alcohol produced from plant cellulosic materials (summarized in FIG. 1). Such alcohols can be produced from purified carbohydrates, such as those found in the kernels of maize or the sugar liquor of sugarcane; however, the cellulosic components of the cell walls of other cell types, which have little or no value as a food commodity, are also a rich source of carbohydrate.

Processes are available that use either acidic or basic conditions and heat to “soften” cellulosic material to allow the carbohydrate subunits to be accessed and released by enzymes such as cellulase, which can degrade cellulose into its component sugars. The component sugars may then be used in a variety of other processes such as in the production of biofuels by fermentation. The cellulose-degradation activity for many of these enzymes has not been measured, and thus it is unknown whether some enzymes have better activity than others for a given cellulosic substrate. Characterizing the activity of enzymes for a particular cellulosic substrate can be useful for increasing the efficiency of the system for producing the component sugars, and thus, increasing the product produced using the component sugars.

Pyruvate is a key intermediate in the production of alcohols from cellulose and/or cellulosic sugars. Specifically, pyruvate is produced during glycolysis by the dephosphorylation of phosphoenolpyruvate. Pyruvate is also a key intermediate in metabolism, and may be used in both anabolic and catabolic reactions. Because pyruvate is created in so many cellular reactions, it is relatively non-toxic to most cell types even at relatively high levels within a cell. Cells can also secrete pyruvate into their environment. Advantageously, pyruvate is stable in solution or as dry solids, which makes their transport safe and non-hazardous. Once at the process destination, pyruvate obtained from cellulosic material may be used to produce alcohol for use as a biofuel alternative.

Many enzymes used by fermentative microbes to convert pyruvate into butanol have been identified (FIG. 6, left panel). Microbial production of alcohol by fermentation suffers from the disadvantage that the resultant alcohol can reach levels that are toxic to the microbes, eventually resulting in death of the fermentive microbes.

While many enzymes and methods for producing alcohol using cellulase-like enzymes have been described, these methods may rely on sub-optimal enzyme activity because of the source of cellulosic material, and thus, result in less alcohol production. Moreover, alcohol production by microbial fermentation processes may be limited by the toxicity of the alcohol toward the microbes used in the fermentation process. Accordingly, a need exists for quantifying enzyme activity for cellulose degradation. Once identified, appropriate enzymes may be matched with a source of cellulosic material to increase the level of pyruvate produced. The pyruvate may then be used as an intermediate in the production of biofuels. Additionally, the conversion of pyruvate to butanol in a cell-free system has not, to the inventors' knowledge, been previously described.

### SUMMARY OF THE DISCLOSURE

The present disclosure generally relates to detecting cellulose in cellulosic materials and producing alcohol using cellulosic materials. More particularly, the present disclosure relates to methods for producing butanol from pyruvate produced from cellulosic material in a cell-free system. The present disclosure also relates to methods for detecting cellulase activity for the production of pyruvate from cellulosic sources.

In one aspect, the present disclosure is directed to a method of producing butanol in a cell-free system. The method includes contacting a pyruvate solution with enzymes wherein the enzymes are selected from the group consisting of 3-hydroxybutyryl-CoA dehydrogenase, butyryl-CoA dehydrogenase, NADH-dependent butanol dehydrogenase B, acetyl-CoA:formate C-acetyltransferase, pyruvate:ferredoxin 2-oxidoreductase (CoA-acetylating), acetyl-CoA:acetyl-CoA C-acetyltransferase, (S)-3-hydroxybutanoyl-CoA:NADP+ oxidoreductase, (S)-3-hydroxyacyl-CoA:NAD+ oxidoreductase, (3S)-3-hydroxyacyl-CoA hydro-lyase, butanoyl-CoA:electron-transfer flavoprotein 2,3-oxidoreductase, acyl-CoA:NAD+ trans-2-oxidoreductase, acetaldehyde:NAD+ oxidoreductase (CoA-acetylating), oxidoreducatse, pyruvate:[dihydrolipoyllysine-residue acetyltransferase]-lipoyllysine 2-oxidoreductase (decarboxylating, acceptor-acetylating), protein-N6-(dihydrolipoyl)lysine:NAD+ oxidoreductase, acetyl-CoA:enzyme N6-(dihydrolipoyl)lysine S-acetyltransferase, and combinations thereof, and collecting butanol. In another aspect, the method includes contacting pyruvate with a solid phase, wherein the solid phase is coupled with 3-hydroxybutyryl-CoA dehydrogenase, butyryl-CoA dehydrogenase, NADH-dependent butanol dehydrogenase B, and combinations thereof, and collecting butanol.

In another aspect, the present disclosure is directed to methods of producing pyruvate. The method includes culturing at least one microorganism in a liquid culture medium under a hypoxic condition; and collecting pyruvate.

In another aspect, the present disclosure is directed to a method for determining cellulose concentration in a sample. The method includes forming a mixture comprising a sample and Congo red dye; and measuring light emitted from the mixture upon excitation of the mixture with light comprising a wavelength of between about 300 nm to about 380 nm. The method may further comprise determining cellulase activity in the sample.

The disclosure will be better understood, and features, aspects and advantages other than those set forth above will become apparent when consideration is given to the following detailed description thereof. Such detailed description makes reference to the following drawings, wherein:

### BRIEF DESCRIPTION OF THE DRAWINGS

FIG. 1 depicts the metabolic pathway for conversion of cellulosic material to alcohols. Solid lines indicate substrate-to-product conversion by the indicated enzyme; dashed lines indicate regulatory interactions.

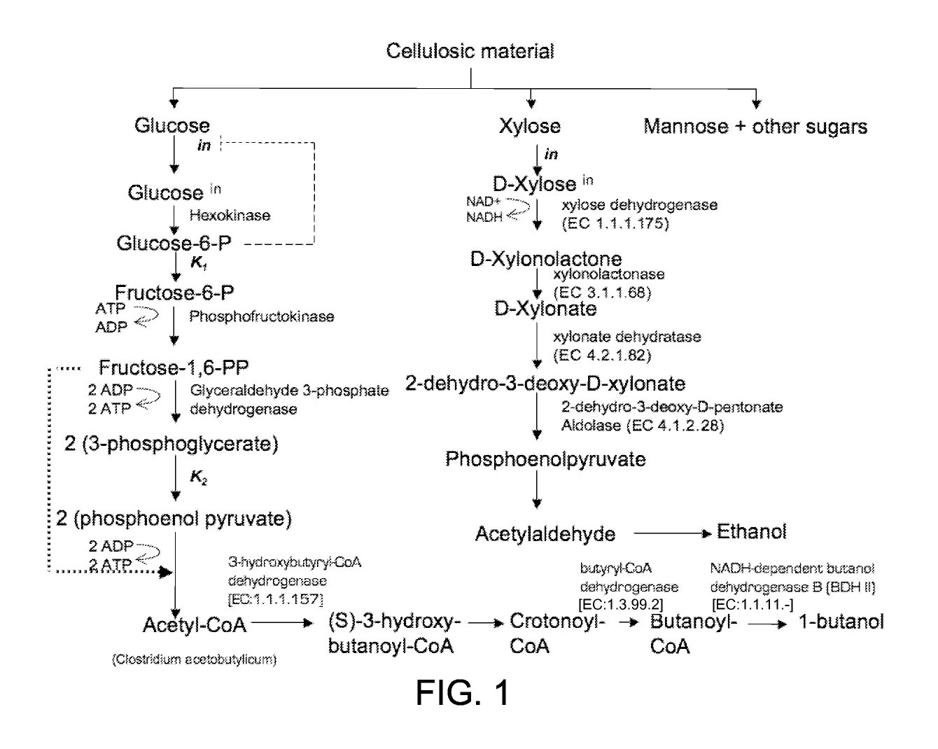

FIG. 2 shows the chemical structure of Congo red dye.

FIG. 3 is a graph showing the absorption spectrum for Congo red dye in aqueous solution as described in Example 3.

FIG. 4 is a graph showing the emission spectrum for Congo red dye in aqueous solution excited at a wavelength 340 nm as described in Example 4.

FIG. 5 is a graph depicting the decrease in the fluorescence of Congo red dye at 420 nm as cellulase is added to a solution containing cellulose as described in Example 5.

FIG. 6 depicts metabolic pathways by which phosphenolpyruvate can be converted into butanol. The left half of the figure shows the pathway as it occurs in fermentative microbes. The right half of the figure shows an alternative, minimal enzymatic pathway (MEP).

FIG. 7 shows the enzyme activity of purified NADH-dependent butanol dehydrogenase as described in Example 9.

FIG. 8 shows the migration of purified NADH-dependent butanol dehydrogenase on a polyacrylamide gel as described in Example 10.

FIG. 9 is an illustration of the apparatus used for culturing Clostridium acetobutylicum as described in Example 6.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, suitable methods and materials are described below.

The present disclosure generally relates to producing alcohol using cellulosic materials. In one aspect, the present disclosure is directed to a method for producing biofuels, and specifically butanol, from pyruvate in a cell-free system. In another aspect, the present disclosure is directed to methods of producing pyruvate from cellulosic material, which may be used in the cell-free system described herein to produce butanol. In another aspect, the present disclosure is directed to methods for identifying cellulose in a sample. The method for identifying cellulose in a sample may be used to determine cellulase activity in the sample. The identification of cellulase activity in a sample may be used to identify and characterize cellulase enzymes useful in the methods for producing pyruvate.

### Method for Producing Biofuels

In one aspect, the present disclosure is directed to a method for producing butanol from pyruvate in a cell-free system. The method includes contacting a solution including pyruvate with enzymes. The enzymes may be directly added to the solution or may be coupled to a solid phase. The enzymes convert pyruvate into butanol following the enzymatic pathway described herein.

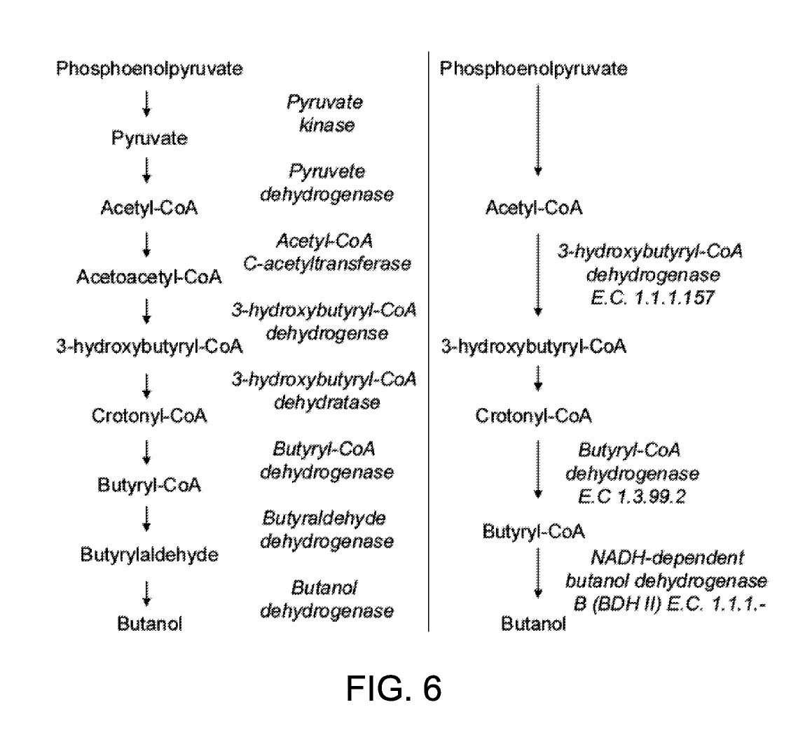

Suitable enzymes that may be used in the cell-free system include those described in Table 1. Based on computational analysis, twenty-seven different combinations of these enzymes may be used in the cell-free system to prepare 1-butanol from pyruvate according to the methods described herein. Particularly suitable enzymes for use in the cell-free system are those that participate in a minimal enzymatic pathway (MEP). The MEP uses the three enzymes 3-hydroxybutyryl-CoA dehydrogenase, butyryl-CoA dehydrogenase, and NADH-dependent butanol dehydrogenase B to convert phosphenolpyruvate to butanol (FIG. 6, right panel).

TABLE 1
Enzymes for Preparing Butanol in a Cell-Free System.

| E.C. Number | Enzyme (Systematic Name) |
|---|---|
| 2.3.1.54 | acetyl-CoA:formate C-acetyltransferase |
| 1.2.7.1 | pyruvate:ferredoxin 2-oxidoreductase (CoA-acetylating) |
| 2.3.1.9 | acetyl-CoA:acetyl-CoA C-acetyltransferase |
| 1.1.1.157 | S)-3-hydroxybutanoyl-CoA:NADP+ oxidoreductase |
| 1.1.1.35 | (S)-3-hydroxyacyl-CoA:NAD+ oxidoreductase |
| 4.2.1.17 | (3S)-3-hydroxyacyl-CoA hydro-lyase |
| 1.3.8.1 | butanoyl-CoA:electron-transfer flavoprotein |
| | 2,3-oxidoreductase |
| 1.3.1.44 | acyl-CoA:NAD+ trans-2-oxidoreductase |
| 1.2.1.10 | acetaldehyde:NAD+ oxidoreductase (CoA-acetylating) |
| 1.1.1.— | Oxidoreducatse able to carry out following reactions: |
| | Butanal + NADH + H+ <=> 1-Butanol + NAD+ OR |
| | Butanal + NADPH + H+ <=> 1-Butanol + NADP+ |
| 1.2.4.1 | pyruvate:[dihydrolipoyllysine-residue acetyltransferase]- |
| | lipoyllysine 2-oxidoreductase (decarboxylating, |
| | acceptor-acetylating) |
| 1.8.1.4 | protein-N6-(dihydrolipoyl)lysine:NAD+ oxidoreductase |
| 2.3.1.12 | acetyl-CoA:enzyme N6-(dihydrolipoyl)lysine |
| | S-acetyltransferase |

Enzymes that are suitable for use in the cell-free system may be selected for a particular activity. For example, 3-hydroxybutyryl-CoA dehydrogenases from Clostridium kluyveri and Clostridium beijerinckii have higher activity by 56.25 and 43.63 fold, respectively, than 3-hydroxybutyryl-CoA dehydrogenase from Clostridium acetobutylicum. Similarly, butanol dehydrogenase from Clostridium acetobutylicum has been found to have 90-fold higher specific activity than butanol dehydrogenase of Clostridium beijerinckii. Enzymes used in the cell-free system may also be isolated from different organisms because of increased expression or ease of isolation and purification. For example, Peptostreptococcus elsdenii has been shown to express 2.76 fold more active butyryl-CoA dehydrogenase than Clostridium acetobutylicum, and thus, may be more suitable source for obtaining butyryl-CoA dehydrogenase for use in the cell-free system.

In one aspect, butanol may be produced by combining all of the components in one mixture. As pyruvate is converted to acetyl-CoA, the acetyl-CoA is converted by 3-hydroxybutyryl-CoA dehydrogenase in the mixture to 3-hydroxybutyryl-CoA, which is then converted to crotonyl-CoA, which is then converted by butyryl-CoA dehydrogenase in the mixture to butyryl-CoA, which is then converted by NADH-dependent butanol dehydrogenase B in the mixture to butanol.

In another aspect, butanol may be produced in sequential reactions. For example, pyruvate may be converted to Acetyl-CoA, which is then contacted with 3-hydroxybutyryl-CoA to produce 3-hydroxybutyryl-CoA. In a separate reaction, 3-hydroxybutyryl-CoA is then converted to crotonyl-Coa, which is then contacted with butyryl-CoA dehydrogenase to produce butyryl-CoA. In another separate reaction, butyryl-CoA is contacted with NADH-dependent butanol dehydrogenase B to produce butanol.

In another aspect, a solution including from about 1 μM to about 1 M of pyruvate may be contacted with a solid phase coupled with the enzymes.

Suitable solid phases may be, for example, polymer beads, glass beads, porous silica, polystyrene particles, alumina particles, structured metal supports, metal oxide particles and combinations thereof.

The enzymes may be coupled to the solid phase by methods known by those skilled in the art (see e.g., Boller, T., et al., “EUPERGIT Oxirane Acrylic Beads: How to Make Enzymes Fit for Biocatalysis,” Org. Proc. Res. Dev. 6(4):509-519 (2002)).

The solution containing pyruvate may be contacted with the enzymes using any suitable method known in the art. One suitable method may be, for example, a batch-type contact whereby the solution is added to the solid phase to form a mixture. Upon contact with the enzyme, the substrate (e.g., pyruvate) is converted to the next product in the enzymatic pathway, which then serves as the substrate for the next enzyme in the enzymatic pathway. Another suitable method may be, for example, to apply the aqueous solution to the solid phase in a column. In one aspect, the solid phase may include a mixture of solid phases having all of the enzymes immobilized thereto. In another aspect, the pyruvate may be sequentially contacted with separate solid phases having the enzymes immobilized thereto.

The cell-free system of the present disclosure allows for the production of purified butanol that does not need to be distilled away from a culture medium when fermentation methods are employed. Additionally, the cell-free system allows for high-yield production of butanol without the problems associated with the toxicity caused by the high alcohol levels in the culture medium.

### Method of Producing Pyruvate

In another aspect, the present disclosure is directed to methods of producing pyruvate. Pyruvate may be derived from cellulose as well as be synthesized by other metabolic pathways. Pyruvate is a key cellular metabolic intermediate that may be generated by the breakdown of glucose via phosphenolpyruvate. More particularly, pyruvate is produced during glycolysis by the dephosphorylation of phosphenolpyruvate.

The method includes culturing at least one microorganism in a liquid culture medium under a hypoxic condition; and collecting pyruvate. As used herein, “hypoxic” refers to any state in which the culture condition (culture medium) comprises less oxygen that it would comprise if it were incubated with agitation by shaking in a loosely capped container. Liquid culture media is often vigorously agitated during incubation to encourage gas exchange between the liquid culture medium and the nearby air. For example, a reference state in which a culture medium would be considered to be normoxic would be 100 ml of culture medium plus 400 ml of atmospheric air contained in a loosely capped 500 ml Erlenmeyer flask.

Any suitable method to lower the amount of oxygen in the liquid culture media may be used to form a hypoxic culture medium, and thus, a hypoxic condition. For example, the culture may be incubated with or without shaking, agitation or aeration. Atmospheric air can be understood to consist of about 20.95% oxygen, as defined by the National Center for Atmospheric Research. Thus, incubating a liquid culture media with atmospheric air having less oxygen than that found in atmospheric air may be understood to cause the liquid culture medium to be hypoxic. Hypoxic culture conditions may also be produced by bubbling gaseous nitrogen (N₂) through the liquid culture medium. Another suitable method to create hypoxic culture conditions may be to allow gaseous nitrogen to displace the atmospheric air above a liquid culture. Yet another method to produce the hypoxic culture condition may be to seal the liquid culture medium, for example, within a tightly capped container. Although the liquid culture medium in a tightly capped container may not initially be hypoxic, over time the culture will become hypoxic as the oxygen in the culture and atmospheric air are consumed.

In some aspects, the culture is maintained in a hypoxic condition when the culture is anoxic. Anoxic conditions may be created by maintaining the culture under an atmosphere flushed with nitrogen gas (N₂). In other aspects, the culture may be maintained in a hypoxic condition when the culture is semi-anaerobic. Semi-anaerobic conditions may be created by sealing a container including the culture and incubating the culture in the sealed container. Semi-anaerobic conditions may also be created by maintaining a culture without agitation. In some configurations, the sealed container, upon sealing, may further include atmospheric air. In some configurations, the sealed container may include a volumetric ratio of atmospheric air:liquid of from about 1:10 to about 1:3. In other suitable configurations, the sealed container may include a volumetric ratio of atmospheric air:liquid of from 1:9 to about 1:4, of from about 1:8 to about 1:4, and of from about 1:7 to about 1:4. In still other suitable configurations, the sealed container may include a volumetric ratio of atmospheric air:liquid of about 1:5.

Suitable microorganisms for use in the method may be, for example, bacteria; fungi; archaea; protists; algae; and animals such as plankton and the planarian. Suitable microorganisms may be, for example, prokaryotes, eukaryotes, and archaebacteria. Suitable prokaryotes may be a gram negative bacterium such as, for example, Escherichia, Salmonella, Shigella, Pseudomonas, Legionella, Wolbachia and Helicobacter; or a gram positive bacterium. Other suitable microorganisms may be, for example, a yeast (fungus), a protist, or an alga. Suitable yeast may be, for example, Saccharomyces, Schizosaccharomyces, Candida, Brettanomyces, Yarrowia, Clostridium, and Cryptococcus. A particularly suitable microorganism is Escherichia coli.

Particularly suitable microorganisms may be those having glycolytic enzymes, and are therefore capable of producing pyruvate. In some aspects, the microorganism may be deficient for at least one enzyme in a metabolic pathway that consumes pyruvate, or for an enzyme that catabolizes pyruvate. Enzymes that may be considered to consume pyruvate include pyruvate oxidase, pyruvate decarboxylase, pyruvate dehydrogenase, dihydrolipoyl transacetylase, dihydrolipoyl dehydrogenase, pyruvate carboyxlase, alanine transaminase, lactate deyudrogenase, citrate synthase, aconitase, isocitrate dehydrogenase, α-ketoglutarate dehydrogenase, succinyl-coA synthethase, succinate dehydrogenase, fumarase, malate dehydrogenase and pyruvate kinase, among others.

In some aspects, the microorganism may be a transformed with an exogenous nucleic acid encoding a transporter protein. Suitable transporter proteins may be, for example, a xylose transporter or a glucose transporter, which move sugars such as glucose and xylose into the microbe; and those transporter proteins which move pyruvate out of the microbe. Such transporter proteins may use active transport or passive transport. Other suitable transporter proteins that use active transport may be cotransporter proteins, symporter proteins, or antiporter proteins, as understood by one of skill in the art. Transporter proteins that transport chemicals and molecules by passive transport may enable diffusion or facilitated diffusion.

In another aspect, the microorganism may be deficient for at least one enzyme in a metabolic pathway that consumes pyruvate and/or have decreased activity of one or more enzyme that uses pyruvate as a substrate for a chemical reaction. The microorganism may be deficient for such an enzyme, for example, because the enzyme has a reduced activity as compared to a wild-type microorganism. The reduced activity may be caused by a lower amount of the enzyme in the microorganism as compared to the amount of enzyme in a wild-type microorganism, or because the enzyme in the microorganism is not as processive as an enzyme in a wild-type microorganism. Enzymes that may be considered to utilize pyruvate as a substrate include pyruvate oxidase, pyruvate decarboxylase, pyruvate dehydrogenase, dihydrolipoyl transacetylase, dihydrolipoyl dehydrogenase, pyruvate carboyxlase, alanine transaminase, lactate deyudrogenase, citrate synthase, aconitase, isocitrate dehydrogenase, α-ketoglutarate dehydrogenase, succinyl-coA synthethase, succinate dehydrogenase, fumarase, malate dehydrogenase, pyruvate kinase, and lactate oxidase.

Microorganisms deficient in any of the enzymes utilizing pyruvate as a substrate may be transformed with an exogenous nucleic acid that results in the enzyme deficiency. In addition to or alternatively, microorganisms may be treated using methods for down-regulating and/or silencing genes encoding enzymes that use pyruvate as a substrate. Methods of down-regulation or silencing genes are known to those skilled in the art. For example, protein expression activity may be down-regulated or eliminated using antisense oligonucleotides, protein aptamers, nucelotide aptamers, and RNA interference (RNAi) (e.g., small interfering RNAs (siRNA), short hairpin RNA (shRNA), and micro RNAs (miRNA). Several siRNA molecule design programs using a variety of algorithms are known to those skilled in the art (e.g., Cenix algorithm, Ambion; BLOCK-iT™ RNAi Designer, Invitrogen; siRNA Whitehead Institute Design Tools, Bioinformatics & Research Computing).

Microorganisms may be transformed using a variety of standard techniques known to those skilled in the art. Such techniques may be, for example, viral infection, calcium phosphate transfection, liposome-mediated transfection, microprojectile-mediated delivery, receptor-mediated uptake, cell fusion, electroporation, and the like. The transfected cells may be selected and propagated to provide recombinant host cells having an expression vector stably integrated in the host cell genome or host cells that transiently express the recombinant protein.

Microorganisms may be grown in one or more liquid culture medium. As used herein, “liquid culture medium” may also be referred to as “culture media”, “liquid culture media”, “culture medium”, and “nutrient broth”. The compositions of such liquid culture media may be found in reference manuals and are well known to those skilled in the art.

In some aspects, the liquid culture medium may be a minimal medium. Particularly suitable minimal medium may be M9 medium.

In some aspects, the liquid culture medium may have glucose. Suitable glucose amounts in the liquid culture media may be from about 0.5% to about 3.8% glucose (weight:volume). Other suitable glucose amounts in the liquid culture media may be from about 1% to about 3% glucose (weight:volume). Particularly suitable amounts of glucose in the liquid culture media may be about 3% glucose (weight:volume).

The method further includes collecting the pyruvate. In one aspect, pyruvate is collected from pyruvate stored within the microorganism. To collect internally stored pyruvate, microorganisms are collected. Microorganisms may be collected by methods known by those skilled in the art. For example, microorganisms may be collected by centrifugation, gravity separation, filtration, and combinations thereof. Collected microorganisms may then be lysed by methods known to those skilled in the art. Suitable methods may be, for example, mechanical homogenization, cell lysis, and combinations thereof.

In another aspect, the pyruvate may be collected from the culture medium. It is known that microorganisms may be able to secrete pyruvate into the culture medium. Thus, pyruvate may be collected from the culture medium as well as from internally stored pyruvate. The collected pyruvate may be further isolated and or purified by methods known to those skilled in the art.

### Methods for Determining Cellulose Concentration and Cellulase Activity

In one aspect, the present disclosure is directed to a method for determining cellulose concentration in a sample. The method includes forming a mixture including a sample and Congo red dye. The mixture is then excited with light including a wavelength of from about 300 nm to about 380 nm. The light emitted from the mixture is then measured at a wavelength of from about 410 nm to about 550 nm. A suitable concentration of Congo red dye may be from about 0.5 μM to about 50 μM. A suitable ratio of Congo red dye to cellulose may be from about 0.5 to about 2.0.

In another embodiment, the method may further include measuring cellulase activity in the sample. The method makes use of the reduction of cellulose concentration in a sample. More particularly, Congo red dye (1-Bromo-2,5-bis-(3-hydroxycarbonyl-4-hydroxy)styrylbenzene) binds cellulose in a sample. Upon excitation of the mixture of the sample and Congo red dye, the fluorescence may be used to determine the concentration of cellulose in the sample. Degradation of the Congo red dye fluorescence signal may be used to determine cellulase activity in the sample. Thus, Congo red dye may be used in the method to quantify the amount of cellulose and cellulase activity.

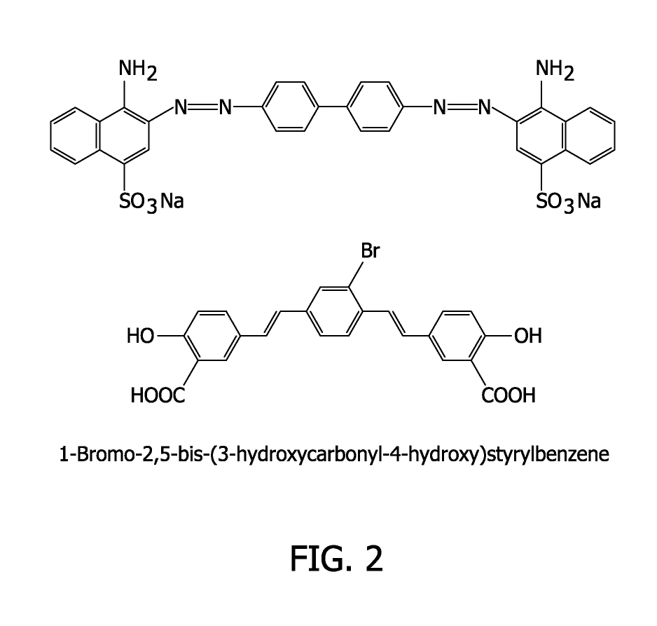

Congo red dye is a hydrophilic dye (shown in FIG. 2). Without being limited by theory, it is believed that Congo red dye stacks within cellulosic fibers. When Congo red dye is exposed to cellulose, it undergoes a shift in fluorescence maximum from 540 nm to 615 nm (excitation at 435 nm).

In one aspect, the method includes exposing the mixture including the sample and Congo red dye to light having wavelengths sufficient to excite the Congo red dye and result in fluorescence. The term “excitation” is used according to its ordinary meaning as understood by those skilled in the art. Suitable excitation wavelengths may be from about 300 nm to about 380 nm. Particularly suitable excitation wavelengths may be from about 320 nm to about 360 nm.

In another aspect, the method includes measuring light emitted from the mixture upon exposure of the mixture to the excitation wavelength. The terms “emitted” and “emission” are used according to their ordinary meaning as understood by those skilled in the art. Suitable emission wavelengths may be from about 420 nm to about 440 nm. A particularly suitable emission wavelength may be about 420 nm.

In another aspect, the pH of the mixture may be from acidic to neutral. Suitable pH of the mixture may be from about 4.8 to about 7.0. Particularly suitable pH of the mixture may be from about 4.8 to about 5.2. A particularly suitable pH of the mixture may be about 5.0.

The sample includes or is suspected of including cellulose. Cellulose is the primary structural component of the cell wall in plants, algae and oomycetes and is a long, straight-chain polymer of D-glucose subunits. About 33% of all plant matter is cellulose. Most plant-derived cellulose usually contains additional substances, such as hemi-cellulose, lignin and pectin.

Cellulose may be difficult to degrade because it readily forms multiple hydrogen bonds between strands that make higher-order structures known as microfibrils, which themselves can form a rigid crystalline matrix. Currently, expensive processes using either hot acid or base are used to loosen the cellulose matrices prior to enzymatic digestion of cellulose into its component sugars.

Suitable sources of cellulose may be any plant biomass. Particularly suitable cellulose sources may be non-food plant biomass. There are many sources of cellulose from non-food plant biomass that are suitable for use in the method of the present disclosure. As used herein, “cellulose” refers to any material that includes cellulose. Cellulose also refers to hemi-cellulose, which is a branched polymer that may include pentose sugars such as, for example, xylose and arabinose, and additional 6-carbon sugars such as, for example, mannose, galactose, and rahamnose. For example, corn kernels are suitable for use in the method, but may be less desirable because of its value as a food. Corn stovers (e.g., stalks and leaves) are an additional source of readily available, cellulose of a non-food plant biomass. Corn stover is made up of three major components: lignin (20%), cellulose (˜45 to 55%) and hemi-cellulose (˜20 to 30%). Other examples of suitable cellulose sources from plant biomass may be raw, prepared or processed switchgrass; waste paper; algae; oomycetes; cotton; wood pulp, such as that from Salix (willow), pine, or Populus (poplar); industrial hemp; Miscanthus; and other plant biomass.

As used herein, the term “cellulase” refers to any enzyme that degrades cellulose into its component D-glucose subunits. Cellulases may also degrade hemi-cellulose into glucose and xylose, arabinose, mannose, galactose, and rhamnose. The activity of a cellulase enzyme on a given substrate may be identified by measuring the amount of cellulose in a sample comprising cellulose at a given time, adding a mixture comprising cellulase enzyme, and measuring the amount of cellulose in the sample at least one later time point. The activity of the cellulase on the cellulose comprised by the sample may be calculated from such a procedure. Cellulases may have varying activity on different cellulosic substrates; cellulase activity may also vary depending on non-cellulose components of the sample. Non-cellulose components that may affect cellulase activity may include, for example, the amount of solvent; the pH; the temperature; salts; and impurities.

In some aspects, the method further includes of measuring cellulose concentration at a first time point and measuring cellulose concentration at least one additional time point. The difference between the cellulose concentration measured at the first time point and the cellulose concentration measured at the additional time point(s) may be used to identify cellulase activity in the sample. Once identified in a sample, the cellulase may be used to convert cellulose into pyruvate according to the methods described herein.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those skilled in the art should, in light of the present disclosure, appreciate that many changes can be made in the specific embodiments that are disclosed and still obtain a like or similar result without departing from the spirit and scope of the invention.

### Example 1

In this Example, cellulose in an aqueous solution was detected using Congo red dye.

A mixture was prepared by mixing 10 μg of cellulose in 5% acetic acid and 0.15 M NaCl. The pH of the solution was 5.0. Congo red dye (5 μM) was added to the mixture and incubated for 60 minutes. The mixture was excited at 340±20 nm and the emission was measured from 370 nm to 700 nm.

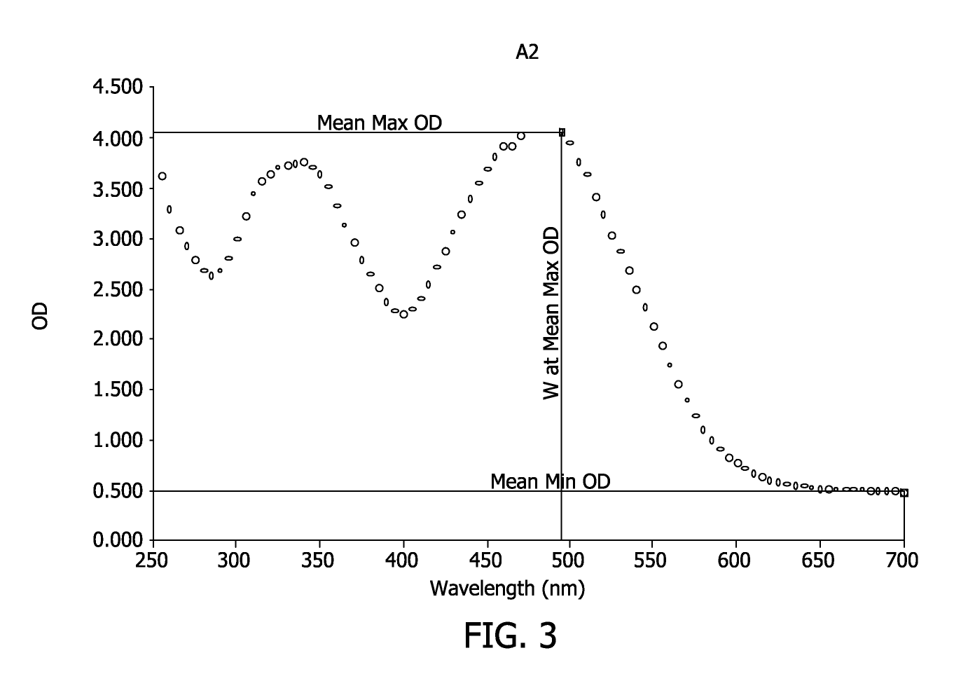

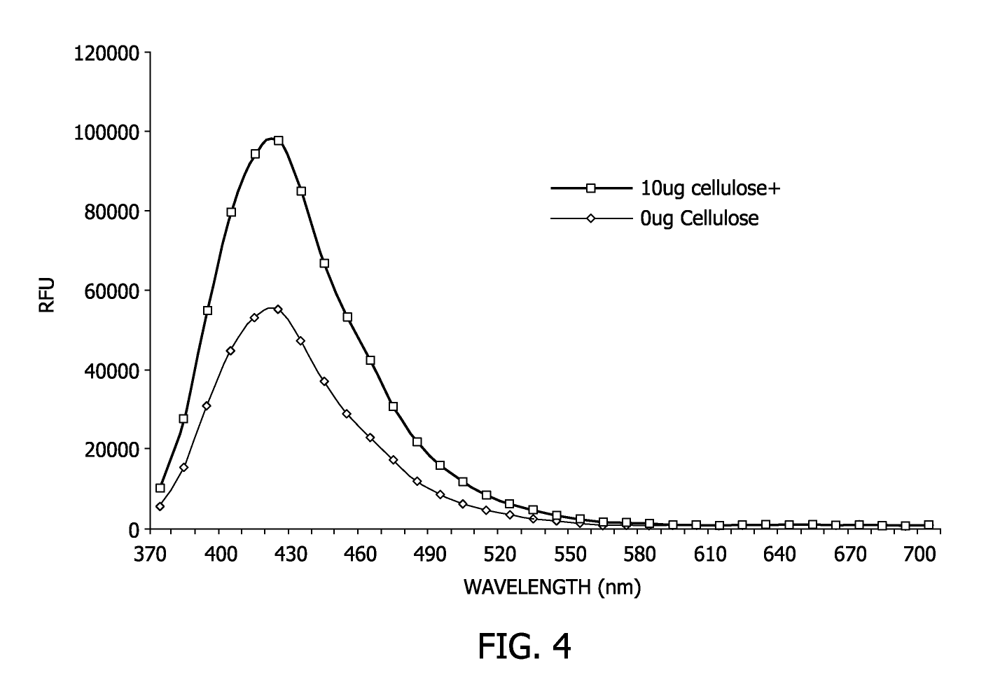

As shown in FIG. 3, Congo red dye has two absorption peaks, one at about 340 nm and a second at about 470 nm. When a solution containing Congo red dye without cellulose was excited at 340±20 nm, an emission spectrum such as that shown in FIG. 4 (⋄ line) was obtained. The emission maximum (˜60000 RFU) occurs at about 420-440 nm (⋄ line). This maximum increased by two- to three-fold (to ˜100000 RFU) when 10 μg cellulose was added to the solution (□ line).

This experiment demonstrated that Congo red dye can be used to measure cellulose concentrations.

### Example 2

In this Example, screening for cellulase activity was determined by detecting cellulose using Congo red dye.

Using the method of measuring cellulose described in Example 1, presence of cellulase activity in an aqueous solution having cellulose was measured. A solution of 10 mg/L cellulose was divided into 5 samples of 45 μl. 0, 1, 5, 10, or 20 U of cellulase (to final volume of 50 μl) was added to separate samples and incubated at 37° C. for 60 minutes. Cellulose concentration was then measured by exciting the sample at 340±20 nm, and reading the emission at 370 to 550 nm.

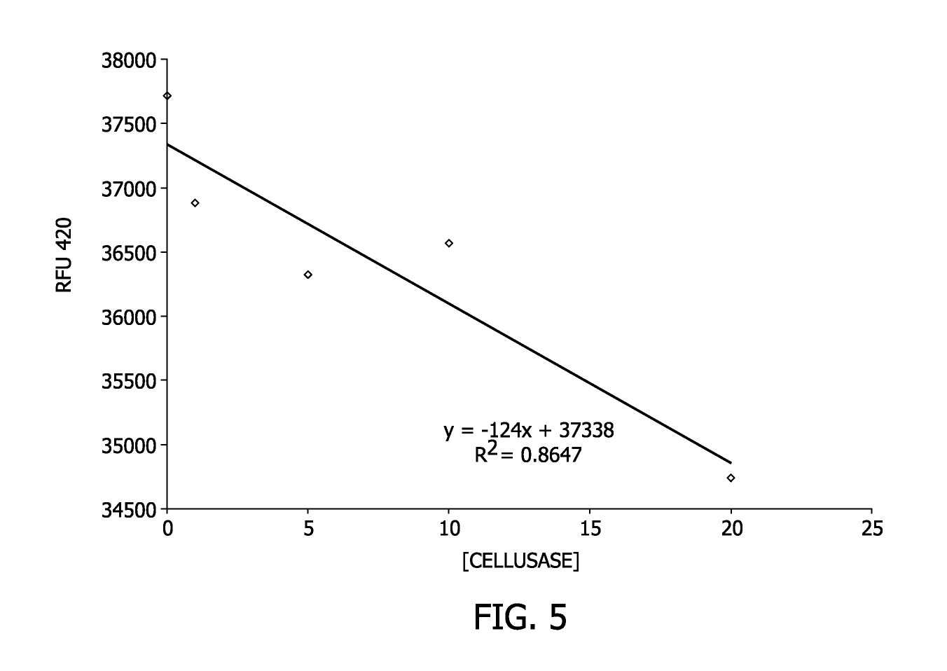

A graph showing results from a representative experiment is shown in FIG. 5. Increasing concentrations of cellulase decreased emission at 420 nm, demonstrating that treatment of cellulose with cellulase decreased the amount of cellulose.

These experiments demonstrated that cellulase activity can be measured using Congo red dye.

### Example 3

In this Example, a method of producing intracellular pyruvate is described.

E. coli was cultured for 24 hours in 2 ml of 3% glucose/M9 growth medium at 37° C. with agitation (under normoxic conditions) or without agitation (under hypoxic conditions). Pyruvate concentration (indicated as [Pyruvate]) was measured by colorimetric assay (BioVision Inc.) using pyruvate as a standard. Pyruvate concentration in some samples was verified by GC-MS (gas chromatography-Mass spectroscopy).

E. coli cultured with agitation under hypoxic conditions or under normoxic conditions produced 45.9 mM and 2.7 mM respectively of pyruvate over 24 hours (Table 1). In contrast, E. coli cultured under semi-aerobic conditions produced 139.7 mM of pyruvate over 24 hours (Table 1).

TABLE 1
Pyruvate yield of E. coli grown as indicated

| | | Lysate (0.05 ml) | | Media (2 ml) | | Total yield per | Calculated yield |
|---|---|---|---|---|---|---|---|
| | Agitated | [Pyruvate] | Yield | [Pyruvate] | Yield | 2 ml culture | per liter |
| Condition | growth | (10⁻⁹ molar) | (10⁻⁶ moles) | (10⁻⁶ molar) | (10⁻⁶ moles) | (10⁻⁶ moles) | (10⁻³ moles) |
| Aerobic | yes | 54 | 2.7 | 0 | 0 | 2.7 | 1.35 |
| Anaerobic | yes | 198 | 9.9 | 18. | 36 | 45.9 | 23 |
| Semi- | yes | 233 | 11.7 | 64. | 128. | 139.7 | 64 |
| Anaerobic | | | | | | | |
| Aerobic | no | 49 | 2.5 | 5.3 | 11.0 | 13.5 | 6.8 |
| Anaerobic | no | 11.2 | 0.56 | 2.0 | 4.0 | 4.56 | 2.3 |
| Semi- | no | 304 | 15.2 | 112 | 224 | 239.2 | 120 |
| Anaerobic | | | | | | | |

Agitated growth indicates if the culture was grown in a shaking incubator (250 RPM; indicated by “yes”) or held stationary (indicated by “no”) at 37° C. in 3% glucose + M9 salts. Results shown are from a 24 hour time point. The GC-MS results show excellent correlation (<5% difference) with the results from the colorimetric assay.

This experiment demonstrated that intracellular pyruvate concentrations in E. coli can be influenced by growth conditions.

### Example 4

In this Example, a method of increasing the concentration of pyruvate in the culture medium is described.

E. coli were cultured in 3% glucose/M9 growth medium at 37° C., as described above. However, in these experiments, the culture was maintained in an anaerobic state by degassing the media, then bubbling nitrogen through media to displace air in both media and the air column above the media. The tubes were capped tightly to prevent loss of nitrogen. Pyruvate concentration in the cells and culture medium was measured by colorimetric assay (BioVision Inc.) using pyruvate as a standard.

When agitation was provided, the total yield of pyruvate in a 2 ml culture after 24 hours was 2.7 mM when bacterial was cultured with agitation (normoxic conditions). When no agitation was provided, 13.5 mM of pyruvate was produced (Table 1).

### Example 5

In this Example, a method of increasing the concentration of pyruvate in the culture medium is described.

E. coli were cultured for 24 hours in semi-anaerobic conditions in 3% glucose/M9 growth medium at 37° C. with or without shaking. The amount of pyruvate in the cells and culture medium was measured as described above.

As shown in Table 1, a 2-ml culture grown in semi-anaerobic conditions without shaking produced 239.2 mM of pyruvate after 24 hours. In comparison, a 2-ml culture grown in semi-anaerobic conditions with shaking produced 139.7 mM of pyruvate after 24 hours. Thus, E. coli cultured without shaking under semi-anaerobic conditions increased pyruvate concentration.

### Example 6

In this Example, Clostridium acetobutylicum ATCC® 824™ were cultured for the isolation of enzymes.

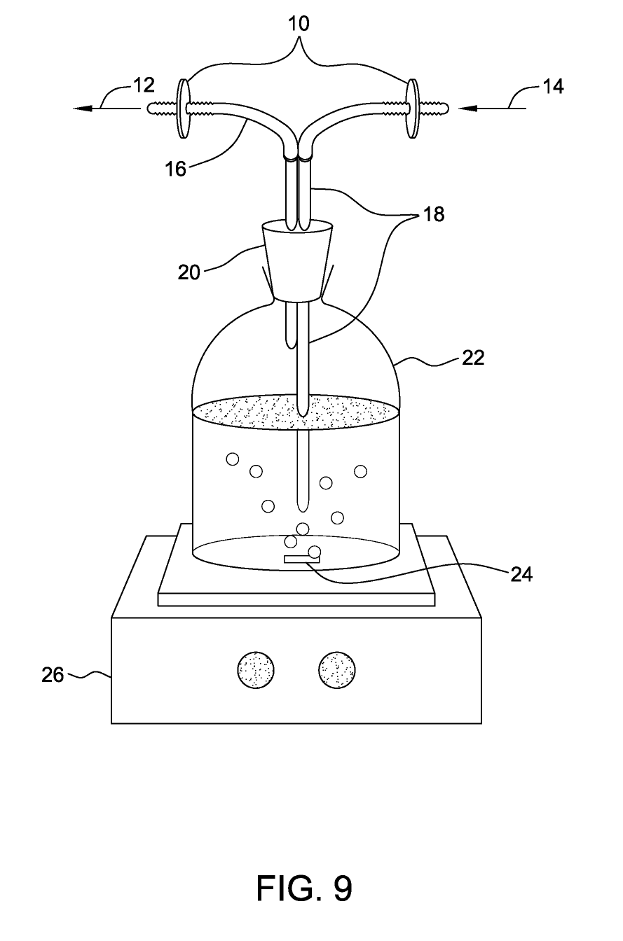

Clostridium acetobutylicum ATCC® 824™ cells were cultured under anaerobic conditions at 37° C. Anaerobic conditions were created using continuous flow of nitrogen gas sterilized by passage through Aervent® 50 Cartridge filter (Millipore) 10. The temperature was controlled by a Revco Environmental Chamber (Thermal Product Solution). The fermentor was a 2 L Pyrex° media bottle 22 sealed with a two-hole rubber stopper 20. The two-hole rubber stopper 20 allowed gas inlet 14 and outflow 12. Nitrogen gas was introduced into the culture medium through an inlet tube 18 and exhausted from the bottle 22 through an outlet tube 16. The bottle 22 was placed on top of a magnetic stirring plate 26 and a stir bar 24 was used to maintain a homogeneous suspension of cells. See e.g., FIG. 9.

Clostridium acetobutylicum ATCC® 824™ spores were hydrated and propagated with 57 g/L cooked meat medium (CMM; beef heart 30 g/L, D(+)-glucose 2 g/L, meat peptone 20 g/L, NaCl 5 g/L (Fluka), yeast extract 3 g/L (Sigma), L-cysteine hydrochloride monohydrate 0.5 g/L (Sigma), sodium acetate 3.0 g/L (Sigma), K₂HPO₄ 5.0 g/L (Fluka), and tryptone 5.0 g/L (Sigma)).

CMM was first inoculated with Clostridium acetobutylicum ATCC® 824™. The Clostridium acetobutylicum ATCC® 824™ was then subjected to a series of glucose medium inoculations to induce the expression of solvent producing enzymes that allow the bacterium to withstand the alcoholic environment. The glucose medium contained (per 1 L): 50.0 g D-(+)-glucose (Sigma), 0.75 g KH₂PO₄ (Sigma), 0.75 g K₂HPO₄ (Fluka), 0.40 g MgSO₄.7H₂O (Sigma), 0.01 g MnSO₄.H₂0 (Sigma), 0.01 g FeSO₄.7H₂O (Sigma), 1.0 g NaCl (Sigma), 2.0 g (NH₄)₂SO₄ (Sigma), 5.0 g yeast extract (Sigma), 0.5 g L-cysteine hydrochloride monohydrate 0.5 g/L (Sigma), 0.003 g 4-aminobenzoic acid (Sigma), 0.00045 g biotin (Sigma).

### Example 7

In this Example, experiments to identify growth conditions that allow for optimized isolation of MEP enzymes are described.

Several growth protocols were used to determine which growth conditions might be most effective for the isolation of MEP enzymes produced by Clostridium acetobutylicum ATCC® 824™. These protocols used are referred to herein as growing protocol (GP) A, B, and C (GP-A, GP-B and GP-C).

GP-A is described in Besic and Minteer (Am. Chem. Soc. Div. Fuel Chem. 54:178-179 (2009)). The organism used was Clostridium acetobutylicum strain ATCC® 824™ (from Manassas, Va.). The spores were hydrated and propagated with the cooked meat medium (CMM) before any solvent inducing inoculations. Cooked meat medium is composed of cooked meat broth 57 g/L (30 g/L beef heart, 2 g/L D(+)-glucose, 20 g/L meat peptone, and 5 g/L NaCl) (Fluka), 3 g/L yeast extract (Sigma), 0.5 g/L L-cysteine hydrochloride monohydrate (Sigma), 3.0 g/L sodium acetate (Sigma), 5.0 g/L K₂HPO₄ (Fluka), 5.0 g/L tryptone (Sigma)). The cooked meat medium was first inoculated to get C. acetobutylicum to grow and replicate followed by a glucose medium inoculation to induce the activity of solvent producing enzymes to withstand the unnatural alcoholic environment. Components of glucose media per 1 L solution: 50.0 g D-(+)-glucose (Sigma), 0.75 g KH₂PO₄ (Sigma), 0.75 g K₂HPO₄ (Fluka), 0.40 g MgSO₄.7H₂O (Sigma), 0.01 g MnSO₄.H₂0 (Sigma), 0.01 g FeSO₄.7H₂O (Sigma), 1.0 g NaCl (Sigma), 2.0 g (NH₄)₂SO₄ (Sigma), 5.0 g yeast extract (Sigma), 0.5 g L-cysteine hydrochloride monohydrate 0.5 g/L (Sigma), 0.003 g 4-aminobenzoic acid (Sigma), 0.00045 g biotin (Sigma).

GP-B used the same meat and glucose media, but the cells were allowed to induce expression of solvent-producing enzymes by growth for up to 4 generations from glucose media instead of a single generation as in GP-A. The first generation defined as the 1^{st} growth in glucose media after the meat media, while the 2^{nd} generation is 2^{nd} growth in glucose media after the 1^{st} growth in glucose media, etc. Each generation was inoculated with cells from previous growth. The 4^{th} generation was added to a fresh glucose media at pH 5.8 and collected right before or at the beginning of the solvent phase when the pH had dropped to 4.8 after 10 hours of fermentation.

GP-C used the same media as GP-A, but the meat media additionally contained 5× L-cysteine, and the glucose media additionally contained (1.0 g/L ZnSO₄, 2.0 g/L asparagine, and 5× more of L-cysteine, 4-aminobenzoic acid and biotin). The cells used in GP-C were in the 2^{nd} generation after growth in glucose media. The medium was not controlled for acid/solvent phase, but was allowed to ferment for a total of 52 hours at which point the cells were deep in the solvent phase.

### Example 8

This Example describes methods used in cell lysis during MEP enzyme extraction.

Cells grown using the GP-A growth protocol described above, were frozen after growth. For enzyme extraction, frozen cells were thawed, lysed and collected according to the procedure in Besic and Minteer (Am. Chem. Soc. Div. Fuel Chem. 54:178-179 (2009)). Frozen cell pellets were suspended in lysis buffer solution composed of 15 mM potassium phosphate buffer at pH 7.0 with 1 mM dithiothrieol (DTT) and 0.1 mM ZnSO₄ (0.5 g of frozen cells per 10 ml of lysis buffer). Lysozyme (50 μl of 1% enzyme solution suspended in 0.3 M potassium phosphate buffer) with sodium deoxycholate solution (135 μl of 10% w/v) were added to the lysis buffer/cell solution and stirred at 4° C. for 1 hour. Then the cell suspension was additionally lysed with a 550 Sonic Dismembrator from Fisher Scientific at level 10 for one pulse of 0.5 seconds. The extract was then centrifuged at 11000 rpm for 1 hour in a Centrifuge 5804-R 15amp from Eppendorf to remove the cell debris and unlysed cells.

Cells grown using GP-B were collected as described above and subsequently lysed with 4 pulses of sonication before the addition of 3× more of lysozyme and additional digester DNase (25 mg/g cell) for 1 hour.

Cells grown using GP-C were not lysed with a sonicator, but with 100 pulls of a homogenizer and 20 hours of DNase and lysozyme (25.0 mg and 12.5 mg/g cell) treatment.

For all the procedures, the cell debris was removed by centrifugation at 3000 g for 1 minute. According to Durre et al. (Appl. Microbiol. Technol. 26:268-272 (1987)), the NADH-dependent butanol dehydrogenase is sedimented at high speeds; therefore, a low-speed spin was used to prevent its removal from the solution).

In all cases, the supernatant from the final spin was assayed for enzymatic activity.

### EXAMPLE 9

This Example describes assays that can be performed on isolated MEP enzymes.

Enzyme activity assays were performed as described in Besic and Minteer (Am. Chem. Soc. Div. Fuel Chem. 54:178-179 (2009)).

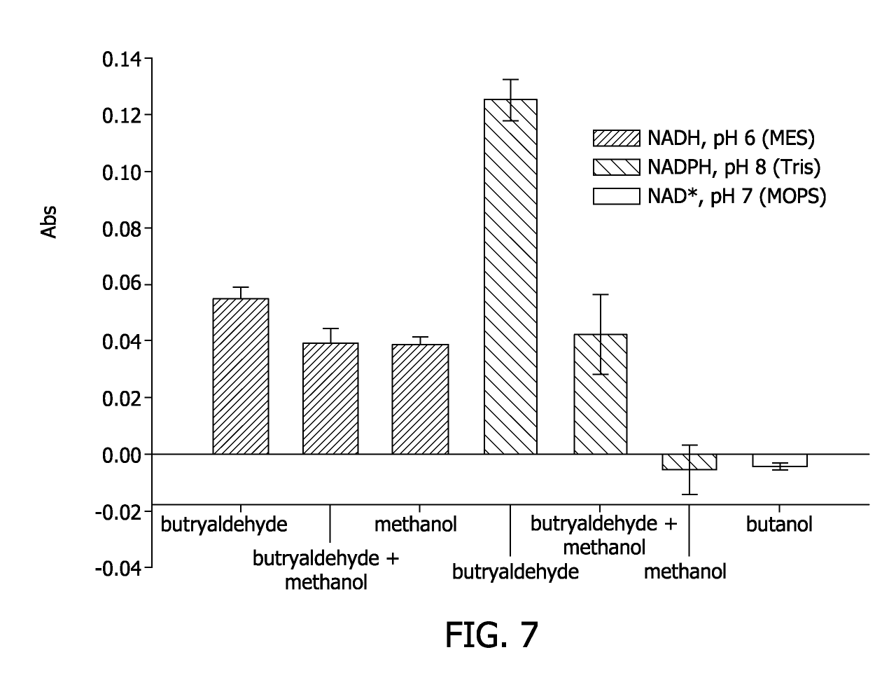

Activity assays were performed at initial temperature of 30° C. with final incubation at room temperature (20-25° C.) on a Genesys 20 spectrophotometer from Thermo Electron Corporation by following the oxidation of either β-nicotinamide adenine dinucleotide (NADH) to NAD⁺ or β-nicotinamide adenine dinucleotide phosphate (NADPH) to NADP⁺ at 340 nm. NADH activity was tested in 50 mM 2-(N-morpholino)ethanesulfonic acid (MES) at pH 6.0 while the activity of NADPH was tested in 50 mM tris(hydroxymethyl)aminomethane (Tris) at pH 8.0 with very low concentrations of coenzymes. For each enzyme, activity was tested in the presence of 75 mM of solvent: butyraldehyde, methanol, and mixture of both (see e.g., FIG. 7). The reaction was initiated with addition of 100 μl of crude extract into 1 ml of coenzyme/solvent buffer solution. Activity in the butanol oxidation direction was determined in 50 mM 3-(N-morpholino)propanesulfonic acid (MOPS) buffer at pH 7.0 with 75 mM butanol and very low concentration of NAD⁺ in which the reaction was initiated with addition of 100 μL of the crude extract.

These experiments demonstrated that MEP enzymes purified from Clostridium acetobutylicum ATCC® 824™ can catalyze oxidation reactions.

### Example 10

This Example describes experiments that were performed to isolate and purify the MEP enzyme NADH-dependent butanol dehydrogenase.

The purification protocol was similar to Welch et al. (Arch. Biochem. Biophys. 273:309-318 (1989)), but 2 grams of cells was used as starting material.

Ion exchange chromatography was performed in a manner similar to Welch et al. (Arch. Biochem. Biophys. 273:309-318 (1989)). However, the DE-52 anion-exchange column was packed and equilibrated using 25 mM potassium phosphate buffer at pH 7.5 with 1 mM dithiothrietol (DTT) and 0.1 mM ZnSO₄, to enhance the capacity of the column.

Unwanted proteins were removed by washing the column using 25 mM potassium phosphate buffer at pH 7.5 with 1 mM DTT and 0.1 mM ZnSO₄. The NADH- and NADPH-dependent butanol dehydrogenase was eluted with the same buffer but the pH was lowered to 6.9 and 1 M NaCl was added.

For affinity chromatography, the NADH- and NADPH-dependent butanol dehydrogenase was specifically eluted with its cofactor NADH (20 mM) and all eluted samples were concentrated with an Amicon concentrator while the phosphate buffer was 10 mM at pH 8 with DTT and ZnSO₄.

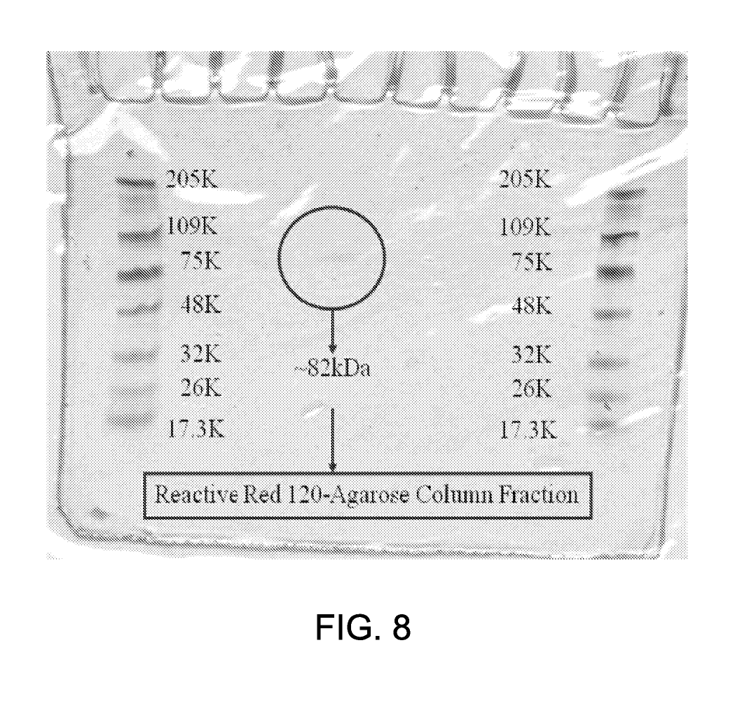

Purification efficiency and molecular weight of NADH-dependent butanol dehydrogenase was determined by 12% polyacrylamide gel electrophoresis. The running buffer was 0.1% of sodium dodecyl sulfate (SDS), 25 mM TRIS and 192 mM glycine at pH 8.3. Molecular weight standards were from Pierce (Blue Protein Molecular Weight Marker Mix prestained: myosin (205K), phosphorylase B (109K), BSA (75K), ovalbumin (48K), carbonic anhydrase (32K), trypsin inhibitor (26K), lysozyme (17.3K)). After electrophoresis, the proteins in the gel were first immobilized with a solution that contained 20% ammonium sulfate and 3% phosphoric acid and stained for 5 days with Coomassie blue dye solution that contained 10% ammonium sulfate, 3% phosphoric acid, 50% methanol, 10% acetic acid and 0.2% Coomassie blue dye. The gel was destained with dH₂O and preserved with ethanol/glycerol mix. See e.g., FIG. 8.

The specific activities of two other enzymes of the MEP were tested in a purified DE-52 fraction using a similar protocol for NADH-dependent butanol dehydrogenase where they all followed the oxidation of NADH at 340 nm. Butyryl-CoA dehydrogenase reacted with crotonyl-CoA while the 3-hydroxybutyryl-CoA dehydrogenase reacted with acetoacetyl-CoA.

Activity assays were performed at initial temperature of 30° C. with final incubation at room temperature (20-25° C.) on a Genesys 20 spectrophotometer (Thermo Electron Corporation) by following the oxidation of either β-nicotinamide adenine dinucleotide (NADH) to NAD⁺ at 340 nm. NADH activity was tested in 50 mM 2-(N-morpholino)ethanesulfonic acid (MES) at pH 6.0. For each enzyme, activity was tested in the presence of 75 mM of crotonyl-CoA or acetoacetyl-CoA. The reaction was initiated with addition of 100 μl of crude extract into 1 ml of coenzyme/solvent buffer solution. Activity in the oxidation direction was determined in 50 mM buffer with 75 mM substrate and very low concentration of NAD⁺ in which the reaction was initiated with addition of 100 μL of the crude extract.

The Examples described above demonstrate that the methods according to the present disclosure offer the ability to produce butanol from pyruvate in a cell-free system. Additionally, the methods of the present disclosure for identifying cellulose in a sample may be used to determine cellulase activity in the sample. The identification of cellulase activity in a sample may be used to identify and characterize cellulase enzymes useful in the methods for producing pyruvate. Methods using cellulosic material to produce pyruvate may be used in the cell-free system described herein to produce butanol.

In view of the above, it will be seen that the several advantages of the disclosure are achieved and other advantageous results attained. As various changes could be made in the above devices and methods without departing from the scope of the disclosure, it is intended that all matter contained in the above description and shown in the accompanying drawings shall be interpreted as illustrative and not in a limiting sense.

What is claimed is:

1. A method of producing butanol in a cell-free system, the method comprising:
contacting an aqueous solution of pyruvate with enzymes, wherein the enzymes are selected from the group consisting of 3-hydroxybutyryl-CoA dehydrogenase, butyryl-CoA dehydrogenase, NADH-dependent butanol dehydrogenase B, acetyl-CoA:formate C-acetyltransferase, pyruvate:ferredoxin 2-oxidoreductase (CoA-acetylating), acetyl-CoA:acetyl-CoA C-acetyltransferase, (S)-3-hydroxybutanoyl-CoA:NADP+ oxidoreductase, (S)-3-hydroxyacyl-CoA:NAD+ oxidoreductase, (3S)-3-hydroxyacyl-CoA hydro-lyase, butanoyl-CoA:electron-transfer flavoprotein 2,3-oxidoreductase, acyl-CoA:NAD+ trans-2-oxidoreductase, acetaldehyde:NAD+ oxidoreductase (CoA-acetylating), oxidoreducatse, pyruvate:[dihydrolipoyllysine-residue acetyltransferase]-lipoyllysine 2-oxidoreductase (decarboxylating, acceptor-acetylating), protein-N6-(dihydrolipoyl)lysine:NAD+ oxidoreductase, acetyl-CoA:enzyme N6-(dihydrolipoyl)lysine S-acetyltransferase, and combinations thereof; and
collecting butanol.

2. The method of claim 1, wherein the enzymes comprise a combination of 3-hydroxybutyryl-CoA dehydrogenase, butyryl-CoA dehydrogenase, NADH-dependent butanol dehydrogenase B.

3. The method of claim 1, wherein the solution of pyruvate is contacted with 3-hydroxybutyryl-CoA dehydrogenase to produce 3-hydroxybutyryl-CoA;
contacting 3-hydroxybutyryl-CoA with butyryl-CoA dehydrogenase to produce butyryl-CoA; and
contacting butyryl-CoA with NADH-dependent butanol dehydrogenase B to produce butanol.

4. The method of claim 1, wherein at least one enzyme is coupled to a solid phase, wherein the solid phase is selected from the group consisting of a polymer bead, a glass bead, porous silica, a polystyrene particle, an alumina particle, a structured metal support, a metal oxide particle and combinations thereof.

5. A method of producing pyruvate, the method comprising:
culturing at least one microorganism in a liquid culture medium under a hypoxic condition; and
collecting pyruvate.

6. The method of claim 5, wherein the hypoxic condition is selected from the group consisting of an anoxic condition, a nitrogen atmosphere, and a semi-anaerobic condition.

7. The method of claim 5, wherein the at least one microbe comprises glycolytic enzymes.

8. The method of claim 5, wherein the at least one microbe is deficient in at least one enzyme of a metabolic pathway that consumes pyruvate.

9. The method of claim 8, wherein the at least one enzyme of a metabolic pathway that consumes pyruvate is at least one enzyme that catabolizes pyruvate.

10. The method of claim 9, wherein the at least one enzyme of a metabolic pathway that consumes pyruvate is selected from the group consisting of pyruvate oxidase, pyruvate decarboxylase, pyruvate dehydrogenase, dihydrolipoyl transacetylase, dihydrolipoyl dehydrogenase, pyruvate carboyxlase, alanine transaminase, lactate dehydrogenase, citrate synthase, aconitase, isocitrate dehydrogenase, a-ketoglutarate dehydrogenase, succinyl-coA synthethase, succinate dehydrogenase, fumarase, malate dehydrogenase, pyruvate kinase, and lactate oxidase.

11. The method of claim 6, wherein the semi-anaerobic condition comprises sealing a container comprising the culture to form a sealed container and incubating the culture in the sealed container.

12. The method of claim 6, wherein the semi-anaerobic condition comprises culturing without agitation.

13. The method of claim 11, wherein the sealed container further comprises ambient air.

14. The method of claim 13, wherein the sealed container comprises a volumetric ratio of ambient air to liquid of from about 1:10 to about 1:3.

15. The method of claim 5, wherein the collecting pyruvate comprises extracting pyruvate from cells, collecting pyruvate from the liquid culture medium, and combinations thereof.

16. A method of determining cellulose concentration in a sample, the method comprising:
forming a mixture comprising a sample and Congo red dye; and
measuring light emitted from the mixture upon excitation of the mixture with light comprising an excitation wavelength of between about 300 nm to about 380 nm.

17. The method of claim 16, wherein the light emitted comprises a wavelength of from about 420 nm to about 440 nm.

18. The method of claim 16, wherein the mixture has a pH of less than 7.0.

19. The method of claim 16, wherein the mixture has a pH of from about 4.8 to about 7.0.

20. The method of claim 16, wherein the sample comprises a plant biomass.
